(19) United States
(12) Patent Application Publication
(10) Pub. No.: US 2012/0010216 A1
(43) Pub. Date: Jan. 12, 2012

(54) Pharmaceutical compositions containing vanoxerine

(76) Inventor: Arthur M. Brown, Cleveland, OH (US)

(21) Appl. No.: 12/801,960

(22) Filed: Jul. 6, 2010

(51) Int. Cl. A61K 31/495; A61P 25/28; A61P 9/06; A61P 25/36

(52) U.S. Cl. 514/255.04

(57) ABSTRACT

Disclosed embodiments are related to compositions of vanoxerine (GBR 12909), including compositions of vanoxerine and one or more diluents, disintegrants, binders and lubricants, and the processes for their preparation thereof.

### FIELD OF THE INVENTION

Presently disclosed embodiments are related to pharmaceutical compositions of vanoxerine and processes for the preparation thereof. Presently disclosed embodiments particularly relate to pharmaceutical compositions that include vanoxerine and one or more diluents, disintegrants, binders and/or lubricants.

### BACKGROUND

Vanoxerine (1-[2-[bis(4-fluorophenyl)methoxy]ethyl]-4-(3-phenylpropyl)piperazine), its manufacture and/or certain pharmaceutical uses thereof are described in U.S. Pat. No. 4,202,896, U.S. Pat. No. 4,476,129, U.S. Pat. No. 4,874,765, U.S. Pat. No. 6;743,797 and U.S. Pat. No. 7,700,600, as well as European Patent EP 243,903 and PCT International Application WO 91/01732, each of which is incorporated herein by reference in its entirety.

Vanoxerine has been used for treating cocaine addiction, acute effects of cocaine, and cocaine cravings in mammals, as well as dopamine agonists for the treatment of Parkinsonism, acromegaly, hyperprolactinemia and diseases arising from a hypofunction of the dopaminergic system. (See U.S. Pat. No. 4,202,896 and WO 91/01732.) Vanoxerine has also been used for treating and preventing cardiac arrhythmia in mammals. (See U.S. Pat. No. 6,743,797 and U.S. Pat. No. 7,700,600.)

It is desirable to optimize the formulation of a solid dose form of vanoxerine, particularly for human use.

The newly discovered formulations preferably use a minimal number of excipients and use pharmaceutical grade excipients that are inexpensive, readily available, and that facilitate cost-effective manufacture on a commercial scale.

### SUMMARY

Embodiments of the present disclosure relate to novel compositions of vanoxerine. In particular, vanoxerine is admixed with various excipients to formulate a solid dose of vanoxerine. In certain embodiments, the solid dose is in tablet form; in other embodiments, it is in capsule form.

An additional aspect of the present disclosure includes processes for the preparation of vanoxerine formulations. In particular, the processes involve preparation of a solid dosage form of vanoxerine, preferably by wet mixing vanoxerine and excipients with water, followed by drying and milling of the granulated mixture.

Other aspects of the present disclosure include use of these compositions for the treatment of a disease or disorder in a subject in need thereof comprising administering to the subject a therapeutically effective amount of the compositions of the presently disclosed embodiments.

### DETAILED DESCRIPTION OF PREFERRED EMBODIMENTS

All references cited herein are hereby incorporated by reference in their entirety.

As used herein, the term “about” is intended to encompass a range of values ±10% of the specified value(s). For example, the phrase “about 20” is intended to encompass ±10% of 20, i.e. from 18 to 22, inclusive.

As used herein, the term “vanoxerine” refers to vanoxerine and pharmaceutically acceptable salts thereof.

As used herein, the term “pharmaceutically acceptable” refers to those compounds, materials, compositions, and/or dosage forms which are, within the scope of sound medical judgment, suitable for contact with the tissues of and/or for consumption by human beings and animals without excessive toxicity, irritation, allergic response, or other problem complications commensurate with a reasonable benefit/risk ratio.

As used herein, the term “subject” refers to a warm blooded animal such as a mammal, preferably a human or a human child, which is afflicted with, or has the potential to be afflicted with one or more diseases and conditions described herein.

As used herein, “therapeutically effective amount” refers to an amount which is effective in reducing, eliminating, treating, preventing or controlling the symptoms of the herein-described diseases and conditions. The term “controlling” is intended to refer to all processes wherein there may be a slowing, interrupting, arresting, or stopping of the progression of the diseases and conditions described herein, but does not necessarily indicate a total elimination of all disease and condition symptoms, and is intended to include prophylactic treatment.

As used herein, “unit dose” means a single dose which is capable of being administered to a subject, and which can be readily handled and packaged, remaining as a physically and chemically stable unit dose comprising either vanoxerine or a pharmaceutically acceptable composition comprising vanoxerine.

Preferred embodiments include pharmaceutical compositions of vanoxerine with one or more excipients, such as those pharmaceutically acceptable diluents, disintegrants, binders and lubricants known and available to those skilled in the art. Preferably, the excipients meet the standards of the National Formulary (“NF”) and/or United States Pharmacopoeia (“USP”). In a particular preferred embodiment, there is provided a pharmaceutical composition comprising vanoxerine with one or more diluents, disintegrants, binders and/or lubricants.

In certain preferred embodiments, the composition comprises vanoxerine; a diluent such as lactose; a binder such as microcrystalline cellulose; a disintegrant such as croscarmellose sodium; a flowing agent such as colloidal silicon dioxide; and a lubricant such as magnesium stearate.

The excipients are selected to ensure the delivery of a consistent amount of vanoxerine in a convenient unit dosage form and to optimize the cost, ease and reliability of the manufacturing process. All excipients must be inert, organoleptically acceptable, and compatible with vanoxerine. The excipients used in a solid oral formulation commonly include fillers or diluents, binders, disintegrants, lubricants, antiadherents, glidants, wetting and surface active agents, colors and pigments, flavoring agents, sweeteners, adsorbents, and taste-maskers.

Diluents are typically added to a small amount of the active drug to increase the size of the tablet. A suitable diluent for use in the inventive compositions is lactose, which exists in two isomeric forms, alpha-lactose or beta-lactose, and can be either crystalline or amorphous. Various types of lactose include spray dried lactose monohydrate (such as Super-Tab™), alpha-lactose monohydrate (such as Fast Flo®), anhydrous alpha-lactose, anhydrous beta-lactose, and agglomerated lactose. Other diluents include sugars, such as compressible sugar NF, dextrose excipient NF, and dextrates NF. A preferred diluent is lactose monohydrate (such as Fast Flo®). Other preferred diluents include microcrystalline cellulose (such as Avicel® PH, and Ceolus™), and microfine cellulose (such as Elcema®).

Suitable diluents also include starch and starch derivatives. Starches include native starches obtained from wheat, corn, rice and potatoes. Other starches include pregelatinized starch NF, and sodium starch glycolate NF. Starches and starch derivatives can also function as disintegrants. Other diluents include inorganic salts, including, but not limited to, dibasic calcium phosphate USP (such as Di-Tab® and Emcompress®), tribasic calcium phosphate NF (such as Tri-Tab® and Tri-Cafos®), and calcium sulfate NF (such as Compactrol®). Polyols such as mannitol, sorbitol, and xylitol may also serve as diluents. Many diluents can also function both as disintegrants and as binders, and these additional properties should be taken into account when developing particular formulations.

Disintegrants may be included to break larger particles, such as tablets, granules, beads, nonpareils and/or dragrees, into smaller particles comprising the active pharmaceutical ingredient and, optionally, other excipients which may facilitate dissolution of the active ingredient and/or enhance bioavailability of the active ingredient. Starch and starch derivatives, including cross-linked sodium salt of a carboxymethyl ether of starch (such as sodium starch glycolate NF, Explotab®, and Primogel®) are useful disintegrants. A preferred disintegrant is cross-linked sodium carboxymethyl cellulose (such as Croscarmellose Sodium NF, Ac-Di-Sol®). Other suitable disintegrants include, but are not limited to, cross-linked polyvinylpyrrolidone (such as Crospovidone NF) and microcrystalline cellulose (such as Avicel® PH).

Binders may also be used as an excipient, particularly during wet granulation processes, to agglomerate the active pharmaceutical ingredient and the other excipients. In all formulation, whether prepared by wet or dry granulation, a particular binder is generally selected to improve powder flow and/or to improve compactibility. Suitable binders include, but are not limited to, cellulose derivatives, such as microcrystalline cellulose NF, methylcellulose USP, carboxymethycellulose sodium USP, hydroxypropyl methylcellulose USP, hydroxyethyl cellulose NF, and hydroxypropyl cellulose NF. Other suitable binders include polyvidone, polyvinyl pyrrolidone, gelatin NF, natural gums (such as acacia, tragacanth, guar, and pectin), starch paste, pregelatinized starch NF, sucrose NF, corn syrup, polyethylene glycols, sodium alginate, ammonium calcium alginate, magnesium aluminum silicate and polyethylene glycols.

Lubricants may be used, particularly in tablet formulations, to prevent sticking of the ingredients and/or dosage form to the punch faces and to reduce friction during the compression stages. Suitable lubricants include, but are not limited to, vegetable oils (such as corn oil), mineral oils, polyethylene glycols (such as PEG-4000 and PEG-6000), salts of stearic acid (such as calcium stearate and sodium stearyl fumarate), mineral salts (such as talc), inorganic salts (such as sodium chloride), organic salts (such as sodium benzoate, sodium acetate, and sodium oleate) and polyvinyl alcohols. A preferred lubricant is magnesium stearate.

In preferred embodiments, vanoxerine generally comprises from about 20-50% by weight of the pharmaceutical composition, more preferably from about 25-40% and most preferably from about 30-35%.

Preferably, the inventive composition also comprises a diluent which is lactose monohydrate, a binder which is microcrystalline cellulose; a disintegrant which is a cross-linked sodium carboxymethyl cellulose; a flowing agent which is colloidal silicon dioxide, and a lubricant which is magnesium stearate. Suitable amounts of each excipient may be determined empirically by one skilled in the art considering such factors as the particular mode of administration (e.g. oral, sublingual, buccal, etc.), amount of active ingredient (e.g. 50 mg, 60 mg, 80 mg, 100 mg, 150 mg, etc.), particular patient (e.g. adult human, human child, etc.) and dosing regimen (e.g. once a day, twice a day, etc.).

In certain preferred embodiments, the inventive compositions may contain lactose monohydrate (e.g. Fast Flo® #316) from about 30-60% of the composition by weight, more preferably from about 35-50% and most preferably from about 40-45%.

In certain preferred embodiments, the inventive compositions may contain microcrystalline cellulose (e.g. Avicel® PH 102) from about 5-30% by weight of the composition, more preferably from about 10-25% and most preferably from about 15-20% by weight,

In certain preferred embodiments, the inventive compositions may contain cross-linked sodium carboxymethyl cellulose (e.g. Ac-Di-Sol®) from about 0.1-10% by weight of the composition, more preferably from about 0.5-5% and most preferably from about 1-3% by weight,

In certain preferred embodiments, the inventive compositions may contain colloidal silicon dioxide (e.g. Aerosil® A-200) from about 0.02 to about 1% by weight of the composition, more preferably form about 0.1 to about 0.6% and most preferably from about 0.2-0.4% by weight.

In certain preferred embodiments, the inventive compositions may contain magnesium stearate from about 0.02 to about 1% by weight of the composition, more preferably form about 0.1 to about 0.6% and most preferably from about 0.2-0.4% by weight.

Solid dosage forms of vanoxerine can be prepared using any of the methods and techniques known and available to those skilled in the art.

For example, a solid dosage form of vanoxerine can be prepared by wet mixing vanoxerine and excipients with water, drying and milling the granulated mixture. In certain embodiments, the final mixture is compressed into a tablet. In other embodiments, the final mixture is encapsulated.

In particular, the process comprises the steps of: (a) dry blending of vanoxerine and one or more excipients to form a dry mixture; (b) wetting the dry mixture with water, preferably with purified water, to form a wet granulation mixture; (c) drying the wet granulation mixture to form a dried granulation mixture; (d) milling the dried granulation mixture to form a milled granulation mixture; (e) mixing a lubricant in the milled granulation mixture to give a final blended mixture; (f) preparing the final blended mixture in a solid dosage form suitable for oral administration.

In certain preferred embodiments, the final blended mixture is compressed into tablets. In other preferred embodiments, the final blended mixture is enclosed in a capsule.

Specifically, in step (a), vanoxerine is blended with all excipients in the final formulation, other than the lubricant. In particular, vanoxerine is thoroughly dry blended with the diluent(s), disintegrant(s) and binder to form a uniform dry mixture. Blenders appropriate for large scale dry blending include twin shell blenders, double cone blenders, and ribbon blenders. Ribbon blenders have the advantage of being used in continuous-production procedures. High-speed, high shear mixers may also be used and offer the advantage of shorter mixing times. The dry mixture may also be granulated, milled into a fine powder, passed through a mesh screen, or micronized, if necessary. Preferably, the dry blending was performed in high shear granulators.

The resulting dry mixture is then wetted with a wetting agent to form a wet granulation mixture in step (b). The wetting agent is typically added over time, usually from about 1 to about 15 minutes, with continuous mixing. Typically, the wetting agent is added to the blender used in the dry blending step. Preferably the wet granulation is carried out in a high shear granulator. In certain embodiments, the wetting agent is an aqueous-based solution. Preferably, the wetting agent is water without any additional solvents, and in particular, without organic solvents. More preferably, the water is purified water.

The type and amount of wetting agent, rate of addition of wetting agent, and the mixing time influences the structure of the granules. The different types of granules, such as pendular, funicular, capillary, etc., can be manipulated to achieve the desired density, porosity, texture and dissolution pattern of the granules, which in turn, determines the compressibility, hardness, disintegration and consolidation characteristics of the dried mixture.

The wet granulation mixture is then dried in step (c) to form a dried granulation mixture with an appropriate moisture content. In certain embodiments, the drying means include a fluid bed or tray dryers. Fluid bed drying yield shorter drying times, in the range from 1 to 3 hours, while tray drying averages 10 to 13 hours. Preferably, the wet granulation mixture is dried in a fluid bed, for preferably about 1-3 hours. Fluid bed drying has the added advantages of better temperature control and decreased costs. The method of drying, drying time, and moisture content are critical to avoid decomposition, chemical migration, and other adverse physical characteristics of dried mixture which can affect the dosage form performance.

The dried granulation mixture is subsequently milled in step (d) to form a milled granulation mixture. The particle size of the dried granulation mixture is reduced to achieve an appropriate particle size distribution for the subsequent processes. In certain embodiments, milling is achieved using a high shear impact mill (such as Fitzpatrick) or a low shear screening mill (such as Comil). The dried granulation mixture may also be screened to select the desired granule size.

In the next step (e), the lubricant was blended with the dried granulation mixture to give a final blended mixture. In certain embodiments, a V blender or bin blenders are used. A preferred blender is a V-shell PK blender. A gentle blending is preferred, such that each granule covered with the lubricant, while minimizing the breaking up of the granules. Increased breaking of the granules results in fine powder, or “fines”. A high fine content results in variations of weight and density during compression into a tablet, as well as increases the need for cleaning of the compression machinery.

The final blended mixture is then prepared in a solid dosage form suitable for oral administration. Solid dosage forms include tablets, capsules, pills, troches, cachets, and the like. In one embodiment, the final blended mixture is compressed into a tablet. The compression machinery typically contains two steel punches within a steel die cavity. The tablet is formed when pressure is exerted on the dried granulation mixture by the punches in the cavity, or cell.

Tableting machines include single-punch machines, rotary tablet machines, gravity feed, and powder assisted machines. Preferably, gravity feed or powder assisted machines are used. Rotary machines operating at high speeds suitable for large-scale production include double rotary machines and single rotary machines. Tablets can also include sugar-coated tablets, film-coated tablets, enteric-coated tablets, multiple-compressed tablets, controlled-release tablets, tablets for solution, effervescent tablets or buccal and sublingual tablets.

Compressed tablets may be characterized by a number of specifications, including diameter size, shape, thickness, weight, hardness, friability, disintegration time, and dissolution characteristics. The tablets preferably have weights, friability and dissolution rates in accordance with USP standards.

In other embodiments, the final blended mixture is enclosed in capsules, preferably hard gelatin capsules. The hard gelatin capsules are commercially available, and are generally made from gelatin, colorants, optionally an opacifying agent such as titanium dioxide, and typically contain 12-16% water. The hard capsules can be prepared by filling the longer end of the capsule with the final blended mixture, and slipping a cap over the top using mG2, Zanasi, or Höfliger and Karg (H&K) machines.

In an alternative embodiment, the present invention provides for a process of preparing a solid dose form of vanoxerine by dry mixing vanoxerine with the excipients. In certain embodiments, the mixture is compressed into a tablet. In other embodiments, the mixture is encapsulated.

In particular, the process comprises the steps of: (a) dry blending of vanoxerine and one or more excipients to form a dry mixture; (b) mixing a lubricant in the dry mixture to give a final blended mixture; (c) preparing the final blended mixture in a solid dosage form suitable for oral administration.

In certain preferred embodiments, the final blended mixture is compressed into tablets. In other preferred embodiments, the final blended mixture is enclosed in a capsule.

Specifically, in step (a), vanoxerine is blended with all excipients in the final formulation, other than the lubricant. Preferably, vanoxerine is thoroughly dry blended with the diluent(s), disintegrant(s) and a binder to form a uniform dry mixture. Blenders appropriate for large scale dry blending include twin shell blenders, double cone blenders, V blenders or bin blenders. A preferred blender is a V-shell PK blender. High-speed, high shear mixers may also be used. The dry mixture may also be granulated, milled into a fine powder, passed through a mesh screen, or micronized, if necessary.

In the next step (b), the lubricant was blended with the dry mixture to give a final blended mixture. In certain embodiments, a V blender or bin blenders are used. A preferred blender is a V-shell PK blender.

The final blended mixture is then prepared in a solid dosage form suitable for oral administration. Solid dosage forms include tablets, capsules, pills, troches, cachets, and the like. In one embodiment, the final blended mixture is compressed into a tablet. In another embodiment, the final blended mixture is enclosed in capsules, preferably hard gelatin capsules.

Other aspects of the invention also include use of these compositions for the treatment of a disease or disorder in a subject in need thereof comprising administering to the subject a therapeutically effective amount of the compositions of the present invention. In particular, the present compositions are useful in the treatment of cocaine addiction, acute effects of cocaine, cocaine cravings, Parkinsonism, acromegaly, hyperprolactinemia and diseases arising from a hypofunction of the dopaminergic system, and cardiac arrhythmia.

### EXAMPLES

The materials, methods, and examples presented herein are intended to be illustrative, and not to be construed as limiting the scope or content of the invention. Unless otherwise defined, all technical and scientific terms are intended to have their art-recognized meanings.

### Example 1

Formulation of a 100 mg Vanoxerine Capsule

| | Amount per | Amount per |
|---|---|---|
| Components | tablet (mg) | batch (mg) |
| GBR 12909 | 100.0 | 120.0 |
| (Vanoxerine) | | |
| Lactose | 121.00 | 145.20 |
| Monohydrate, NF | | |
| Microcrystalline | 51.00 | 61.20 |
| Cellulose, NF | | |
| Croscarmellose | 6.00 | 7.20 |
| Sodium, NF | | |
| Colloidal Silicon | 1.00 | 1.20 |
| Dioxide, NF | | |
| Magnesium | 1.00 | 1.20 |
| Stearate, NF | | |
| Total Tablet | 300.0 | 336.0 |
| Weight | | |

### Example 2

Formulation of a 200 mg Vanoxerine Capsule

| | Amount per | Amount per |
|---|---|---|
| Components | tablet (mg) | batch (mg) |
| GBR 12909 | 200.0 | 240.0 |
| (Vanoxerine) | | |
| Lactose | 242.00 | 290.40 |
| Monohydrate, NF | | |
| Microcrystalline | 102.00 | 122.40 |
| Cellulose, NF | | |
| Croscarmellose | 12.00 | 14.40 |
| Sodium, NF | | |
| Colloidal Silicon | 2.00 | 2.40 |
| Dioxide, NF | | |
| Magnesium | 2.00 | 2.40 |
| Stearate, NF | | |
| Total Tablet | 600.0 | 672.0 |
| Weight | | |

### Example 3

Large Scale Preparation (300 kg) of Vanoxerine Formulation

Step (a): Dry Mixture

Pass Vanoxerine (100.00 kg), Lactose Monohydrate NF (121.00 kg), Microcrystalline Cellulose NF (51.00 kg), Croscarmellose Sodium NF (6.00 kg) and Colloidal Silicon Dioxide (1.00 kg) through a #10 mesh screen. Add the screened material to a 600 liter Collette mixer. Mix for 6 minutes at low speed, without a chopper.

Step (b): Wet Granulation Mixture

To a stainless steel tank, add Purified Water USP (100.00 kg). While mixing the dry mixture at low speed, pump the purified water into the Collette mixer at a rate of 14 kg/min. After the water has been added, continue to mix the wet granulation mixture at low speed and low chopper for 30 additional seconds. Additional mixing, and/or additional water may be required to achieve the desired consistency. Discharge the wet granulation mixture from the Collette bowl into a suitable transport vessel.

Step (c): Drying Wet Granulation Mixture

Spread the wet granulation evenly, and not to exceed 2 inches in depth, on 2 drying racks lined with 40 lb. Kraft paper. Place the racks in G&G Steam Heated Oven. Dry the wet granulation mixture at 60° C.±2° C. until a L.O.D. of 1.0-2.1% is reached.

Step (d): Milling the Dried Granulation Mixture

Pass the dried granulation mixture through an auger feed Fitz® mill (Model DAS06), with knives forward, at medium speed, through a 2 Å screen.

Step (e): Mixing a Lubricant

Add the dried granulation mixture from the previous step to a 20-cubic foot V-shell PK blender (Model C266200). Pass Magnesium Stearate NF (1.00 kg) through a 10-mesh screen into a properly prepared container. Add approximately half of the Magnesium Stearate to each side of the PK blender and blend for 5 minutes.

Step (f): Compression into Tablets

Add the blended granulation mixture form the previous step to Kikusui tablet press for compression into capsule-shaped tablets. The compression equipment can be outfitted to make tooling for a 100 mg tablet (0.496×0.218 inches), a 200 mg tablet (0.625×0.275 inches, bisected), 300 mg tablet (0.715×0.315 inches) and a 400 mg tablet (0.750×0.330 inches).

Alternative Step (f): Filling into Capsules

Add the blended granulation mixture form the previous step to H & K 400 machine for filling the appropriate size capsules.

Although the present invention has been described in considerable detail, those skilled in the art will appreciate that numerous changes and modifications may be made to the embodiments and preferred embodiments of the invention and that such changes and modifications may be made without departing from the spirit of the invention. It is therefore intended that the appended claims cover all equivalent variations as fall within the scope of the invention.

What is claimed is:

1. A pharmaceutical composition in unit dosage form comprising vanoxerine, in an amount of from about 20-50% of the composition by weight; a diluent in an amount of from about 30-60% of the composition by weight; a binder in an amount of from about 15-25% of the composition by weight; a disintegrant in an amount of from about 1-5% of the composition by weight; a flowing agent from about 0.2-0.4% of the composition by weight; and a lubricant from about 0.2-0.4% of the composition by weight.

2. The pharmaceutical composition of claim 1, wherein said diluent is lactose monohydrate.

3. The pharmaceutical composition of claim 1, wherein said binder is microcrystalline cellulose.

4. The pharmaceutical composition of claim 1, wherein said disintegrant is cross-linked sodium carboxymethylcellulose.

5. The pharmaceutical composition of claim 1, wherein said flowing agent is colloidal silicon dioxide.

6. The pharmaceutical composition of claim 1, wherein said lubricant is magnesium stearate.

7. The pharmaceutical composition of claim 1, wherein said vanoxerine is present in an amount of from about 30-35% by weight of the composition.

8. The pharmaceutical composition of claim 2, wherein said lactose monohydrate is present in an amount of from 40-45% by weight of the composition.

9. The pharmaceutical composition of claim 3, wherein said microcrystalline cellulose is present in an amount of from 15-20% by weight of the composition

10. The pharmaceutical composition of claim 4, wherein said cross-linked sodium carboxymethylcellulose is present in an amount of from 1-3% by weight of the composition.

11. The pharmaceutical composition of claim 5, wherein said colloidal silicon dioxide is present in an amount of from 0.2-0.4% by weight of the composition.

12. The pharmaceutical composition of claim 6, wherein said magnesium stearate is present in an amount of from 0.2-0.4% by weight of the composition.

13. The pharmaceutical composition of claim 1, wherein said unit dosage form is a capsule.

14. The pharmaceutical composition of claim 1, wherein said unit dosage form is a tablet.
